(19) United States
(12) Patent Application Publication
(10) Pub. No.: US 2008/0242939 A1
(43) Pub. Date: Oct. 2, 2008

(54) Retractor system for internal in-situ assembly during laparoscopic surgery

(76) Inventor: William Johnston, Evanston, IL (US)

Correspondence Address: IP FOCUS LAW GROUP, LTD, 608 NORTH CARLYLE LANE, SUITE 100, ARLINGTON HEIGHTS, IL, 60004, US

(21) Appl. No.: 12/077,622

(22) Filed: Mar. 20, 2008

Related U.S. Application Data
(60) Provisional application No. 60/909,657, filed on Apr. 2, 2007.

(51) Int. Cl. A61B 1/32

(52) U.S. Cl. 600/204

(57) ABSTRACT

A method of laparoscopic (or robotic) surgery, using a hand-port, comprising providing a trocar port operably disposed within a first abdominal incision opening of a patient, providing a hand-port operably disposed within a second abdominal incision opening, introducing an elongate positioner dimensioned to extend through the trocar, introducing a spatulate element through the hand port and joining the spatulate element to the positioner. The procedure further comprises removing an internal organ or other tissue from the operating area in order to make room and add visibility for the laparoscopic intervention, detaching the spatulate element from the positioner, and withdrawing the positioner through the trocar port and the spatulate through the hand port.

### CROSS-REFERENCE TO RELATED APPLICATION

This patent application is a non-provisional of U.S. provisional patent application Ser. No. 60/909,657, filed Apr. 2, 2007.

### FIELD OF THE INVENTION

The present disclosure is generally directed to surgical instruments and methods and, more particularly, to a surgical instrument and method of internal retraction of tissue, vessels, and organs during laparoscopic surgery and robotic assisted laparoscopic surgery.

### BACKGROUND OF THE INVENTION

Minimally invasive surgical procedures are typically conducted through one or more small ports inserted through relatively small incisions, in contrast to the larger incisions typical of open surgery. Although the proper label varies according to the zone of a patient's body into which instruments will be inserted, the term “laparoscopic” is often used less precisely as a general reference to various types of minimally invasive procedures, e.g., also thoracoscopic surgery.

Laparoscopic surgery specifically involves creating a space by infusing a gas to allow vision and operating through small ports placed though the skin into the space formed. The term “pneumoperitoneum” is used to refer to maintenance of a gas pressurized abdominal cavity during laparoscopic surgery. A camera (laparoscopic camera) placed through an incision port provides vision for the operating surgeon, while other instruments are manipulated. The ports used to retract the incision area and maintain a seal for pneumoperitoneum are typically based on a 10-12 mm instrument diameter platform, though smaller diameters are available for more limited procedures.

Laparoscopic surgery may be robotic assisted. Robotic assisted laparoscopic surgery utilizes mechanical arms and instruments that pass through incision ports and allow greater articulation within the abdomen. The surgeon sits at a workstation that provides three-dimensional vision and “joysticks” to control the mechanical arms, camera, and instruments. A robotic assisted surgery platform is commercially available from Intuitive Surgical, Inc. (Sunnyvale, Calif.) under the designation “daVinci.” A suitable robotic assist system is also described in U.S. Pat. No. 6,770,081 to Cooper et al., the disclosure of which is incorporated herein by reference.

More recently, larger gas-sealed port systems have been developed for the purpose of accommodating a surgeon's hand. In the hand-assisted laparoscopic (HAL) procedures, these ports allow the surgeon to use one hand in conjunction with standard laparoscopic instruments in the other hand. Therefore, hand-assisted, laparoscopic surgery (HALS) combines some of the benefits of both, the open and the laparoscopic methods. In a HALS procedure, a surgeon still places small ports into the abdomen to insufflate, to view and to introduce instruments into the abdominal cavity. In a HALS procedure, however, a surgeon also creates an incision into the abdominal wall large enough to accommodate the surgeon's hand. The intra-abdominal hand provides tactile sensation, three dimensional special orientation, tissue palpation, blunt dissection, retraction and can provide pressure to help gain hemostasis. Laparoscopic hand ports are commercially available from a few different medical equipment companies: “Gelport,” Applied Medical (Rancho Santa Margarita, Calif.), “Omniport,” (Advanced Surgical Concepts, Wicklow, Ireland), “LapDisc,” Ethicon Endosurgery (Cincinnati, Ohio).

Surgeons may perform various procedures laparoscopically where bodily structures must be separated or retracted from surrounding tissue. Although the insufflation gas expands the abdomen to permit the surgeon to view the surgical site, it is often necessary to manipulate the internal organs or tissues to provide a clear path to the surgical objective. Conventionally, small, thin, long instruments are used to perform surgery and retract tissue, vessels, and organs. Examples of these tissue structures include tendons, veins, nerves, arteries, intestines, liver, spleen, and the like. A delicate separation of adjacent tissue structures is often desirable, but can be technically difficult due to the limits of instruments that must fit through the working ports.

Efforts at developing laparoscopic retractor mechanisms which can be used to push and hold the tissue or organs away from the surgical site are reflected in available patent documents. For example, U.S. Pat. No. 4,654,028 to Suma, U.S. Pat. No. 4,909,789 to Taguchi et al., and U.S. Pat. No. 5,195,505 to Josefsen are all directed to collapsible paddles and/or fingers which expand after the retractor has been inserted into the abdomen through the trocar cannula. U.S. Pat. No. 4,190,042 to Sinnreich and U.S. Pat. No. 4,744,363 to Hasson are directed to instruments with collapsible fingers joined by webs of resilient material which expand to form the retractor.

The Cuschieri retractor operates according to the same principle but is instead expanded after insertion by mechanically compressing a multi-segment distal end to force a hook or similar shape.

Despite these developments, tissue retraction remains a challenge in minimally invasive surgical procedures. A need exists for a laparoscopic organ retraction system having sufficient strength and durability to retract body organs from the operative site and, more particularly, for an endoscopic organ retraction system which is relatively small and may be utilized with smaller conventional trocar cannulas to provide access to the site during an endoscopic or laparoscopic (robotic) surgical procedure.

### BRIEF SUMMARY OF THE INVENTION

The methods and related devices disclosed herein overcome the disadvantages associated with the prior art and provide full-duty internal organ or tissue retraction in a laparoscopic procedure. More specifically, a method of hand-assisted laparoscopic surgery according to the present invention comprises providing a trocar port operably disposed within a first abdominal incision opening of a patient, providing a hand-port operably disposed within a second abdominal incision opening, introducing an elongate positioner dimensioned to extend through the trocar, introducing a spatulate element through the hand port, and joining the spatulate element to the positioner. With the spatulate element joined to the positioner, the procedure then comprises removing an internal organ or other tissue from the operating area in order to make room and visibility for the laparoscopic intervention, detaching the spatulate element from the positioner, and withdrawing the positioner through the trocar port and the spatulate element through the hand port.

The positioner has a distal coupling portion and the spatulate element has a corresponding proximal end coupling portion. Each coupling portion is configured for mutual interconnection and they together define a rigid, detachable joint suitable for supporting the weight of an internal organ. The spatulate element has a transverse clearance dimension of at least about 2 centimeters (cm).

Another aspect of the present invention provides a retractor system for use in hand-assisted laparoscopic procedure having a trocar port and a hand port. The system comprises an elongate positioner dimensioned for insertion through the trocar having a distal coupling portion, and a fixed profile spatulate element having a proximal coupling portion and a transverse clearance dimension of at least 2 centimeters (cm), wherein the distal coupling portion and the proximal coupling portion together define a quick-change joint. The distal coupling portion optionally comprises a coupling adapter having a clamp and a distal coupling feature adapted to mate with the coupling portion of the spatulate element.

Another method aspect of the present invention encompasses a method of robotic-assisted laparoscopic surgery in which a spatulate element is introduced through a hand-port or entry port and attached to a positioner that is controlled by robotic arms. Alternatively, the spatulate element may be attached to the robotic instrument and introduced through a trocar-port.

A kit aspect of the present invention includes components for laparoscopic surgery including an elongate positioner dimensioned for insertion through a trocar port and having a distal coupling portion, a plurality of spatulate elements each having a proximal coupling portion and a transverse clearance dimension of at least 2 centimeters (cm). The distal coupling portion of the positioner is removably attachable to the proximal coupling portion of each spatulate element to define detachable joints.

### BRIEF DESCRIPTION OF THE DRAWINGS

FIG. 1 is a schematic elevation view of a surgery patient illustrating the in situ assembly of a laparoscopic retractor according to the present invention;

FIG. 2 is an exploded perspective view of a laparoscopic retractor system according to the present invention;

FIG. 3 is an exploded perspective view of a distal end portion of a positioner and corresponding spatulate element according to an alternate embodiment of the present invention;

FIG. 4 is a enlarged perspective view of a retractor system according to the present invention shown with a trocar port;

FIG.5 is a partial perspective view of the retractor system of FIG. 3 shown in the assembled configuration;

FIG. 6 is a simplified cross sectional view taken generally along the plane 6-6 of FIG. 4 illustrating the details of the detachable coupling;

FIG. 7 is an enlarged side view, partly in section, of the distal end portion of a positioner and corresponding spatulate element according to an alternate embodiment of the present invention;

FIG. 8 is a perspective view of the distal end portion a positioner and corresponding spatulate element according to another alternate embodiment of the present invention;

FIG. 9 is schematic elevation view of a surgery patient illustrating the in-situ shape adjustment of a laparoscopic retractor according to the present invention;

FIG. 10 is a side view, partially in section, of a laparoscopic retractor system according to an alternate embodiment of the present invention in which a mechanical adapter is secured to the distal end portion of a positioner to provide a coupling to the spatulate element;

FIG. 11 is a top plan view of one embodiment of a kit assembly for use in a laparoscopic surgical procedure according to the present invention; and

FIG. 12 is perspective view of a laparoscopic retractor system according to another aspect of the present invention.

### DETAILED DESCRIPTION OF THE INVENTION

The invention disclosed herein is, of course, susceptible of embodiment in many different forms. Shown in the drawings and described here in detail are preferred embodiments of the invention. It is to be understood, however, that the present disclosure is an exemplification of the principles of the invention and does not limit the invention to the illustrated embodiments.

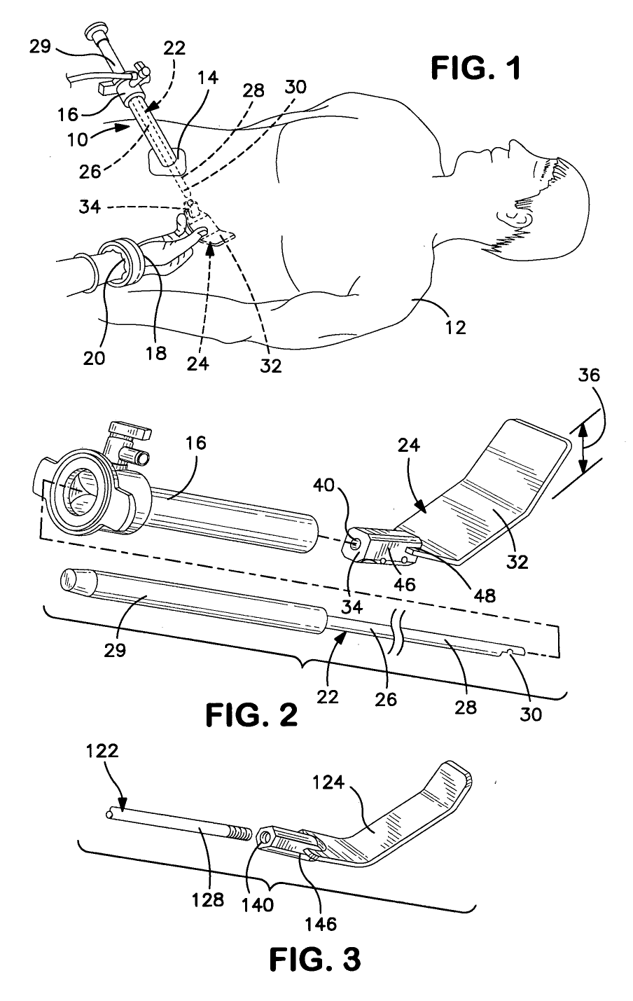

Referring now to FIGS. 1-2, a retractor system 10 according to the present invention is shown schematically in use with a laparoscopic surgery patient 12. The patient 12 bears a first incision opening 14 retracted with a sealing trocar port 16 and a second incision opening 18 retracted with a sealing hand port 20. Retractor system 10 includes a positioner 22 and a spatulate element 24. Positioner 22 has an elongate shaft 26 terminating in a distal end coupling portion 28 with a coupling feature 30 and a proximal or handle portion 29. Positioner 22 is dimensioned for insertion through a laparoscopic trocar port 16. Accordingly, the diameter of positioner shaft 26 is dictated by the instrument diameter platform selected for the surgery but is preferably less than about 12 millimeters (mm), and more preferably less than 10 millimeters (mm).

In contrast, the dimensions for spatulate element 24 are not limited by the opening size or clearance of the laparoscopic trocar. It is a key feature of the present invention that the spatulate element is large enough to securely and reliably retain and/or retract internal organs or tissue. Spatulate element 24 includes a blade portion 32 for retaining internal organs or tissue, and a coupling portion 34 for connection to positioner 22. Spatulate element 24 is relatively large enough to present a surface for contacting and retracting tissue or organs. In this regard, spatulate element 24 has a transverse clearance dimension of at least about 2 centimeters (cm).

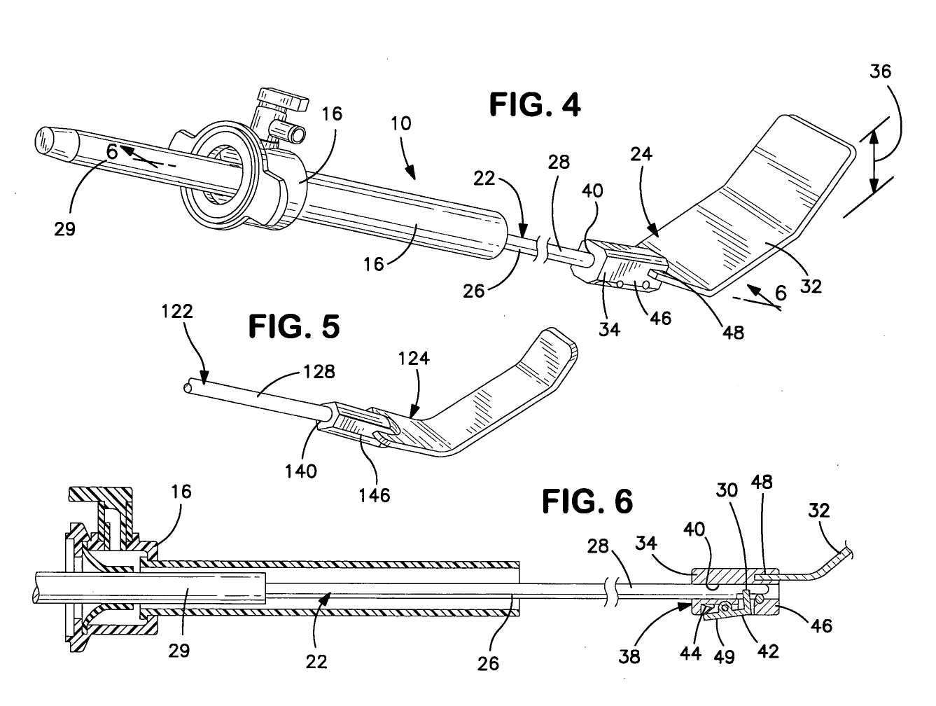

As used herein, the term “transverse clearance dimension” is a reference to the clearance requirement for the spatulate element in any configuration, i.e., the diameter or clearance required of an opening to allow passage of the spatulate element. For example, if the spatulate element was partially collapsible or foldable, the “transverse clearance dimension” is a reference to the clearance requirement of the spatulate element in the smaller collapsed or folded configuration. The spatulate element preferably has a fixed profile, however, as shown in FIGS. 1 through 6. The transverse clearance dimension for spatulate element 24 is identified by reference number 36, and is preferably greater than 2 centimeters (cm), and more preferably greater than 3 centimeters (cm).

It is a further feature of the present invention that spatulate element 24 is readily attachable to positioner 22. In a preferred embodiment as illustrated in FIGS. 1, 2, 4 and 6, spatulate element 24 includes a proximal coupling portion 34 with a latching subassembly 38 adapted to engage a latch catch 30 of distal coupling portion 28 of positioner 22. Latching subassembly 38 comprises a socket 40 for receiving distal coupling portion 28, a latch lever 42 and a latch spring 44. Latch subassembly 38 is defined by and/or contained within a link element 46 which also provides a fixed lap joint 48 with blade element 32. Latch mechanism 38 provides for robust in-situ assembly and disassembly. For assembly, the surgeon may insert the distal end 28 of positioner 22 into socket 40 and spring-based latch lever 42 automatically engages catch 30. For disassembly, the surgeon my actuate lever 42 at position 49 to release latch mechanism 38.

In an alternate embodiment as shown in FIGS. 3 and 5, a positioner 122 is joinable to a spatulate element 124 via a threaded inter-coupling. Positioner 122 includes a threaded distal coupling portion 128, while spatulate element 124 contains a threaded socket 140 defined within a link element 146. While a latch or other quick-action coupling mechanism is generally preferred, a threaded inter-coupling may be desirable to prevent inadvertent disassembly of the joint.

Positioner 22 and spatulate element 24 may be formed of various metallic and non-metallic, e.g., plastic, materials of varying rigidity. Without intending any limitation, among metallic materials of construction, stainless steel and alloys of titanium are preferred. Suitable non-metallic materials include acrylonitrile-butadiene-styrene (ABS) copolymers, polycarbonates, polyurethanes, and the like.

Preferred methods of the present invention allow laparoscopic surgical procedures with improved access to targeted organs or tissue areas. For example, laparoscopic surgeries requiring anterior spine exposure involve substantial efforts to temporarily retract or relocate organs, tissue, and vascular structures. Among other standard steps completed before treatments to the target area, a patient will be prepared with one or more smaller incisions and corresponding trocar ports 16 and at least one larger incision for setup of a sealed handport 20. A laparoscopic camera is introduced through one of the trocar ports 16. Although pneumoperitoneum is preferably established before retractor elements are introduced, the temporary loss of seal when objects are introduced may require some additional insufflation.

With the specific sequence dictated by surgeon preference, a surgeon introduces spatulate element 24 through hand port 20 and positioner 22 through trocar port 16. Via camera assistance and/or tactile feedback or by using other ports and laparoscopic instruments, the surgeon aligns distal end portion 28 of positioner 22 to the proximal coupling portion 34 of spatulate element 24. Latch mechanism 38 engages upon insertion of distal end portion 28 into socket 40 as best shown in FIG. 6. The surgeon next positions retractor system 10 to engage organs or other tissue, and selectively removes organs or other tissue for improved access and visibility to a target site such as a spinal disc space.

Handle portion 29 of positioner 22 may be clamped or otherwise secured outside the patients' body. For example, positioner 22 may be clamped to a positioning system including a flexible arm and clamp. Suitable surgical holding systems are commercially available from Mediflex Surgical Products (Islandia, N.Y.) under the designations “FlexArm” and “StrongArm.”

Following the surgical treatment of the target tissue area, retracted organs and tissue are returned to appropriate pre-surgery positions. Retractor system 10 is then disassembled by the surgeon at joint 38. Again with the specific sequence dictated by surgeon preference, the surgeon removes spatulate element 24 through hand port 20 and positioner 22 through trocar port 16.

It is a key benefit of the present invention that access can be provided in laparoscopic procedures to important treatment sites without the use of specialized access devices. The present invention is likewise compatible with such devices, however. For example, PCT Publication No. WO9730666 to Zdeblick et al., the disclosure of which is incorporated herein by reference, describes specialized laparoscopic instrumentation providing a sealed working channel to the disc space through which the disc space is distracted, vertebral endplates and surrounding discs are reamed, and the vertebral implant inserted, all under direct vision through a laparoscopic port engaged to the end of the sleeve.

Other surgical procedures which may beneficially include the devices and methods of the present invention include disc fusion implantation, pancreatectomy, nissen fundoplication, esophoghealectomy, rectopexy, aortoiliac surgeries such as repair of abdominal aortic aneurism, after removing the internal organ or other tissue form the operating area, and urologic upper tract surgeries (e.g., nephrectomy, partial Nephrectomy, and donor nephrectomy).

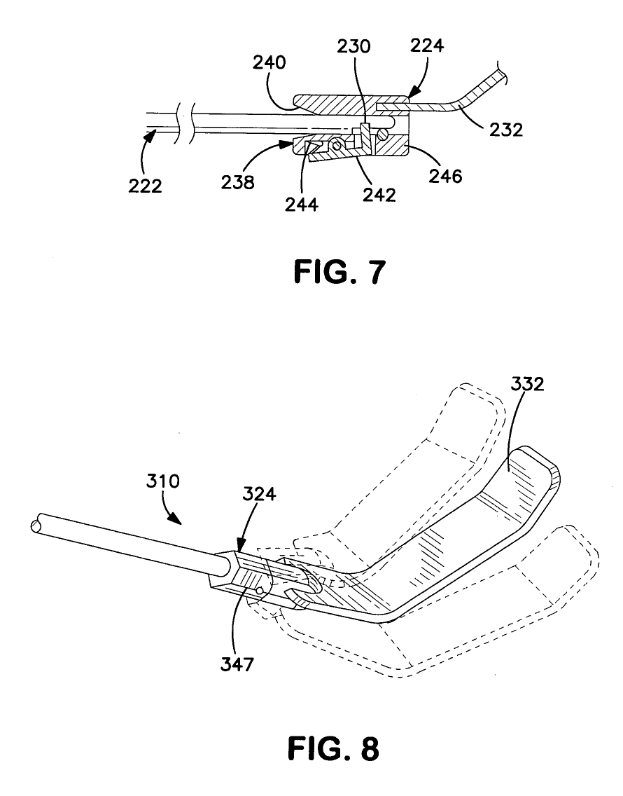

In an alternate embodiment as shown in FIG. 7, a positioner 222 is joinable to a spatulate element 224 via a latch coupling subassembly 238. Positioner 222 includes a distal coupling portion 228 with a latch catch feature 230. Spatulate element 224 contains a tapered socket 240 defined within a link block 246. Latching subassembly 238 comprises a latch lever 242 and a latch spring 244. Latch subassembly 238 is defined by and contained within a link block 246 which also provides a fixed lap joint 248 with blade element 232. Latch mechanism 38 provides for robust in situ assembly and disassembly. For assembly, a surgeon inserts the distal end 228 of positioner 222 into socket 240 such that spring-based latch lever 242 automatically engages catch 230. Tapered, funnel-shaped socket 240 provides a faster alignment, and therefore, more surgeon-friendly in-situ interconnection during HALS procedures.

Shown in FIG. 8 is an embodiment of the present invention which provides adjustment of the angle between the positioner and the retractor blade. Retractor system 310 includes a spatulate element 324 with an articulating joint 347 by which blade 332 is movable with respect to the axial orientation of positioner 322. The methods of the present invention preferably may include providing retractor system with a spatulate element 324 having an articulating joint 347, introducing such a spatulate element 324 through an incision-adaptable sealed port and then actuating joint 347 to a desired incident angle before engaging tissue. The joint actuating step can be completed by the surgeon via the incision-adaptable sealed port within the patient body cavity (i.e., in situ).

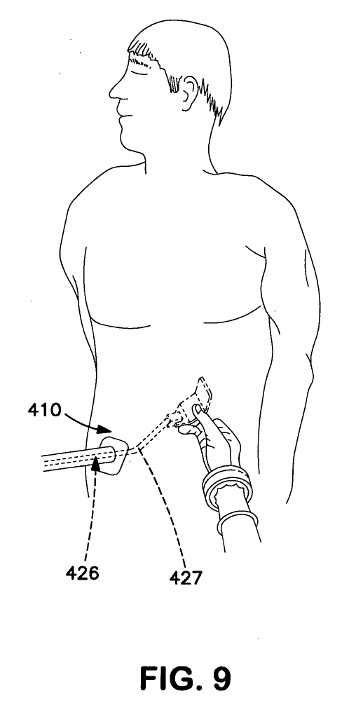

To provide still further in situ adjustability of spatula position and to allow working space for multiple instruments, retractor systems according to the present invention may include a positioner with one or more bendable portions. Referring now to FIG. 9, retractor system 410 includes a positioner shaft 426 with one or more bendable sections 427 that hold a particular shape when bent. Accordingly, the methods of the present invention preferably may include providing a retractor system with a positioner shaft having a bendable section, introducing such a positioner through a body port and then bending the bendable sections to a desired incident angle. The bending step can be completed by the surgeon via the HALS port within the patient body cavity (i.e., in situ) as shown in FIG. 9. Alternatively, all of shaft 426 may be substantially bendable (or malleable).

Malleable section 427 or all of shaft 426 is made from metals, flexible polymers or combinations thereof. Suitable metals include, for example, soft metals, such as soft stainless steel or copper, a cobalt chromium nickel alloy and a nickel-cobalt-chromium-molybdenum alloy. Suitable flexible polymers include elastomers, thermoplastics and other polymers that can sustain significant flexure, bending, twisting and/or deformation without structural failure. Particularly preferred flexible polymer materials include, for example, polyurethane. Generally, the malleable section 427 is bent manually and holds its position in that configuration. Therefore, appropriate materials hold a shape once bent into that shape. The manually induced bend may only occur in a small portion of the malleable section.

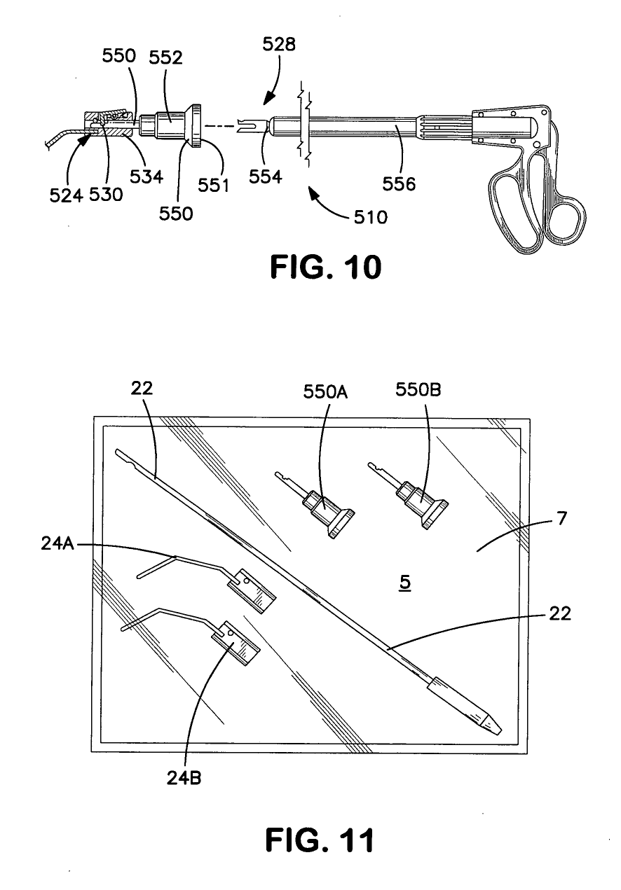

Shown in FIG. 10 is a retractor system 510 in which a distal coupling portion 528 optionally comprises a coupling adapter 550 having a counter-rotation chuck clamp 552 and a distal coupling feature 530 adapted to mate with a coupling portion 534 of spatulate element 524. Coupling adapter 550 preferably defines a receptacle 551 for receiving a distal end portion 554 (and end effector) of a laparoscopic instrument 556. Adaptor 550 includes a chuck clamp 552 for securing end portion 554 within receptacle 551. Retractor system 510 can incorporate a variety of laparoscopic instruments 556 with narrow elongate shaft portions to serve as the positioner. Laparoscopic instruments suitable for serving as a positioner in the present invention include but are not limited to laparoscopic scissors, graspers, forceps, dissectors, clamps, needle holders, clip appliers, fan retractors, cauterization devices and stapling devices.

An important embodiment of the present invention is a surgical kit including a plurality of spatulate elements 24. Each spatulate element 24 is large enough to present a surface for contacting and retracting tissue or organs, and preferably has a transverse clearance dimension of at least about 2 centimeters (cm). An exemplary kit embodiment 5 is illustrated in FIG. 11 and includes a positioner 22, two spatulate elements 24A and 24B, and two coupling adapters 550A and 550B. The positioner 22, spatulate elements 24A and 24B, and coupling adapters 550A and 550B are packaged on a tray 7 comprising a planar central portion. The tray may be formed of any substantially rigid material capable of withstanding conventional sterilization techniques without failure, e.g., a thermoset resin. A cover, such as a clear film, is bonded to the tray, or preferably the tray may be packaged in a wrapping, to allow sterilization of the tray and its contents.

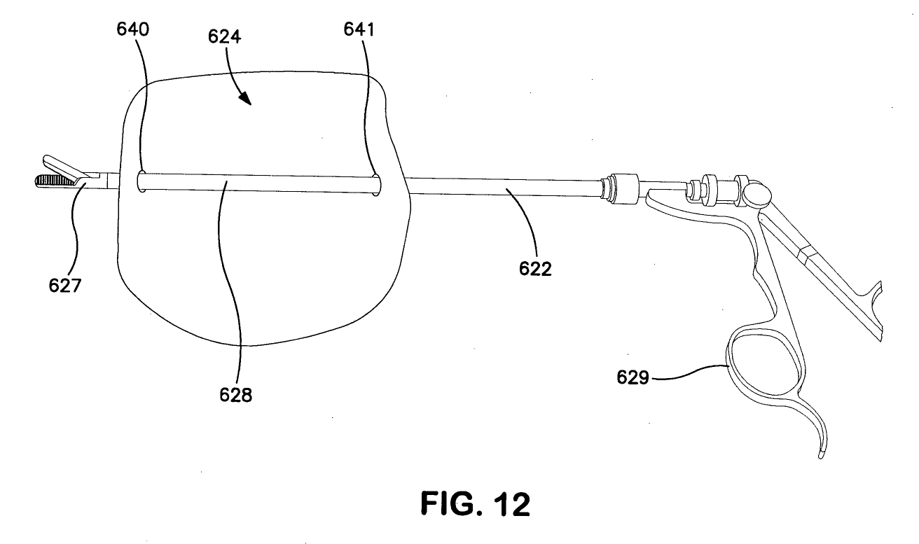

FIG. 12 illustrates an alternate spatulate element 624 that is securable to a positioner 622 through aligned receptacles 640 and 641. Positioner 622 includes scissor style handles 629 to actuate a distal clasp mechanism (not separately shown) which is adapted for clamping to tissue such as an abdominal wall. Spatulate element 624 is especially suited for liver retraction. Positioner 622 preferably takes the form of a conventional laparoscopic instrument having a shaft portion 626 and a conventional end effector such as a clamp 627. Retractor system 610 is constructed in situ by providing both conventional laparoscopic ports (e.g., port 16 shown in FIG. 1) and an incision-adaptable sealed port (e.g., port 20 shown in FIG. 1). Positioner 622 is inserted through the conventional narrow instrument port while spatulate element 624 is introduced through the incision-adaptable sealed port. Via hand-assist or other laparoscopic technique, shaft portion 626 is threaded through openings 640 and 641. In a preferred surgical method, positioner 622 includes a distal clamp 627 and positioner 622 can be set to extend laterally across an abdominal cavity by securing the distal end to firm tissue. In this manner, spatulate element 624 is positioned to remove tissues or larger organs for a target surgical site.

As used herein the term “trocar port” is a reference to a sleeve or collar-like implement which optionally can comprise a piercing tool. The term “trocar” originally referred to a pointed device for penetrating body tissues having a pyramidal point with three faces (from the French trois quarts, meaning three-quarters). By common usage, the term has been broadened to also include devices for placing an access cannula into a body cavity for endoscopic surgery, including laparoscopy, arthroscopy and thoracoscopy. This type of trocar device generally has a cannula, sometimes referred to as a trocar tube, with an obturator within the lumen of the cannula for penetrating the body tissue. A trocar cannula is an elongated hollow tube that functions as a sleeve for the trocar obturator. The trocar cannula may be used as a passage way for the insertion and withdrawal of surgical instruments in laparoscopic surgery. The term trocar port also refers to the assembly of the trocar obturator and trocar cannula. As used herein the term “hand port” is a reference to a size-adaptable sealed access valve or port, which allows for the introduction of relatively larger objects or devices (i.e., >15 mm clearance) into the laparoscopic area such as a surgeon's hand but also allows for the maintenance of pneumoperitoneum during or after such an insertion.

As used herein the phase “hand assisted laparoscopic surgery” or “hand assisted surgery” is a reference to the presence of a size-adaptable sealed access valve or port, which allows for the introduction of relatively larger objects or devices into the abdominal cavity and may include intra-cavity manual (or hand) manipulations. The use of a surgeons hand to introduce larger items through a size adaptable sealed port without the hand entering the abdominal cavity is also contemplated, however.

According to methods of the present invention, relatively larger, spatulate elements are inserted through incision-adaptable ports and then attached to a positioner or other holders which have been inserted through a standard, fixed-dimension laparoscopic port. It is another key feature of the present invention that the spatulate elements can include curved or segmented retraction blades extending circumferentially over 90 degrees or more (.e.g., blade 32, FIG. 2).

Such larger retractor blades, once assembled to the positioner are used to retract vessels and tissue adjacent to the target surgical area, e.g., a spinal area. The methods of the present invention are include laparoscopic vascular repairs in which the in situ assembled retractor systems used to retract adjacent veins or arteries (and portions of larger vessels like aorta or vena cava) to allow exposure for repair of vessels such as in aortic repairs or aortic bypass surgeries. The methods of the present invention further include retraction of vessels during retroperitoneal lymph node dissection in which dissection of lymph node tissue surrounding the great vessels and branches is necessary.

Similarly, the present inventive methods include laparoscopic liver, pancreas, and kidney surgery in which retraction of the great vessels and their branches and/or adjacent organs is usually required. In this regard, it is a feature of the present invention that the in situ assembled retractor systems can reliably secure tissue of relatively higher weights or greater sizes than is possible with conventional laparoscopic retractors which may be inserted through standard ports. As noted above, the present invention provides for retractor blades having greater curvature (e.g., 45, 90 or 120 degrees) as compared to what is available from conventional laparoscopic retractors.

In an alternate method embodiment of the present invention, the spatulate element is inserted through an incision adaptable port and then grasped by another laparoscopic instrument for final assembly such that a surgeon may avoid inserting her hand into the abdominal cavity.

With special reference to the laparoscopic system shown in FIG. 12, a laparoscopic grasping instrument can be used to hold tissue/organs out of the surgical field. However, the small width of the instrument makes it more difficult to maintain such retraction. The retraction efficacy of such grasping instruments is substantially improved by introducing a spatulate element 224 into a surgical cavity and then attaching the spatulate element to a shaft or other feature of the grasping instrument. In this manner, a larger retractor blade is presented for securing tissue. Such retractor systems according to the present invention are well suited to retract such organs as a liver or bowel during kidney surgery.

During robotic surgery, and more specifically during robotic prostatectomy, the bowel and/or bladder often fall down into the pelvis area. This retraction failure interferes with the exposure of the prostate. By attaching or threading a spatulate element onto or around robotically manipulated laparoscopic instrument, the bowel and/or bladder are better secured out of the targeted surgical field. By threading the spatulate element 224 onto a laparoscopic instrument proximally, one retains the working distal end. The distal end of the instrument is then available to grasp tissue while the spatulate element applies pressure proximally to hold the bladder/bowel out of the surgical operative site. During gastric bypass surgery, for example, a spatulate element 224 which is threaded onto the shaft of a laparoscopic instrument allows the instrument to both grasp tissue and retract.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of laparoscopic surgery, the method comprising:
providing a trocar port operably disposed within a first abdominal incision opening;
providing an incision-adaptable sealed port operably disposed within a second abdominal incision opening;
introducing a spatulate element through the incision-adaptable port, the spatulate element having a transverse clearance dimension of at least about 2 centimeters and a proximal coupling portion;
introducing an elongate positioner dimensioned to extend through the trocar port and having a distal coupling portion;
joining the coupling portions of the spatulate element and the positioner to define a tissue retractor;
removing an internal organ or other tissue from the operating area in order to make room and increase visibility for the laparoscopic intervention;
disconnecting the coupling portion of said spatulate element and said positioner; and
withdrawing said positioner and said spatulate element.

2. The method of claim 1 wherein the incision-adaptable port is dimensioned to receive a surgeon's hand.

3. The method of claim 1 wherein the coupling portion of the positioner is joined to the coupling portion of the spatulate element while the spatulate element is grasped in a surgeon's hand.

4. The method of claim 1 further comprising the step of completing a surgical procedure that is a member of the group consisting essentially of disc fusion implantation, pancreatectomy, nissen fundoplication, esophoghealectomy, rectopexy and repair of abdominal aortic aneurism, after removing the internal organ or other tissue from the operating area.

5. The method of claim 1 wherein the step of introducing the spatulate element is completed before the step of introducing the positioner.

6. The method of claim 1 wherein the distal coupling portion comprises a coupling adapter having a clamp and a distal coupling feature adapted to mate with the coupling portion of the spatulate element.

7. The method of claim 1 wherein the distal coupling portion comprises an adapter defining a socket and a distal coupling feature adapted to mate with the coupling portion of the spatulate element.

8. The method of claim 1 further comprising providing a robotic assistance actuator wherein the positioner includes a proximal end portion operably connected to the robotic assistance actuator.

9. The method of claim 1 further comprising providing a flexible arm holder for securing the positioner.

10. A laparoscopic surgical method requiring anterior spine exposure of a patient, the method comprising:
providing a trocar operably disposed within a first abdominal incision opening;
providing a hand-port operably disposed within a second abdominal incision opening;
introducing an elongate positioner dimensioned to extend through the trocar port and having a distal coupling portion;
introducing a spatulate element through the hand port, the spatulate element having a transverse clearance dimension of at least about 2 centimeters and a proximal coupling portion;
joining the coupling portions of the spatulate element and the positioner to define a tissue retractor;
removing an internal organ or other tissue to increase visibility of a spinal disc space;
disconnecting the coupling portion of each said spatulate element and said positioner; and
withdrawing said positioner and said spatulate element.

11. A retractor system for use in a hand-assisted laparoscopic procedure having a trocar port and a hand port, the system comprising:
an elongate positioner dimensioned for insertion through the trocar port and having a distal coupling portion; and
a fixed profile spatulate element having a proximal coupling portion and a transverse clearance dimension of at least 2 centimeters, wherein the distal coupling portion and the proximal coupling portion together define a detachable joint.

12. The retractor system of claim 11 wherein the distal coupling portion comprises a coupling adapter having a clamp and a distal coupling feature adapted to mate with the coupling portion of the spatulate element.

13. The retractor system of claim 12 wherein the clamp is positioned opposite the coupling feature over a length of the coupling adapter.

14. The retractor system of claim 11 wherein the spatulate element is a fixed-profile blade.

15. The retractor system of claim 11 wherein the spatulate element is foldable.

16. A kit assembly according to the present invention for use during laparoscopic surgery with an instrument having an elongate body portion dimensioned for insertion through a trocar port and terminating in distal ends, the kit comprising:
first and second coupling adapters each having a clamp for engaging the elongate body and a distal coupling feature;
first and second spatulate elements each having a proximal coupling portion and a transverse clearance dimension of at least about 2 centimeters, wherein said proximal coupling portion and said distal coupling feature together define a detachable joint.

17. The kit of claim 16 wherein at least one coupling adapter defines a receptacle for receiving the distal end of the instrument.
